(19) United States
(12) Patent Application Publication
(10) Pub. No.: US 2006/0224161 A1
(43) Pub. Date: Oct. 5, 2006

(54) DEPTH GAUGE APPARATUS AND METHODS

(75) Inventor: Timothy Bhattacharyya, Brookline, MA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: THE GENERAL HOSPITAL CORPORATION, Boston, MA (US)

(21) Appl. No.: 11/277,113

(22) Filed: Mar. 21, 2006

Related U.S. Application Data
(60) Provisional application No. 60/667,404, filed on Apr. 1, 2005.

(51) Int. Cl. A61B 17/60

(52) U.S. Cl. 606/102

(57) ABSTRACT

Various methods and devices for measuring the dimensions of a bone hole or cavity are provided. In one exemplary embodiment, a device for measuring the depth of a bone hole includes a depth indicator that is adapted to measure a depth of a bone hole, and a positioning member disposed through the depth indicator for positioning the device relative to the bone hole.

### CROSS-REFERENCE TO RELATED APPLICATIONS

The present application claims priority to U.S. Provisional Application Ser. No. 60/667,404 filed on Apr. 1, 2005 and entitled “Depth Gauge Apparatus and Methods,” which is hereby incorporated by reference in its entirety.

### FIELD OF THE INVENTION

The present invention relates to surgical devices, and in particular to surgical devices for measuring the dimensions of a bone hole or cavity.

### BACKGROUND OF THE INVENTION

Many surgical procedures require the dimensions of a hole to be measured. For example, in orthopedic surgeries involving bone fractures, two or more sections of bone are immobilized by the insertion of a pin or wire therethrough. In order to determine the appropriate length of the pin or wire to be used, it is often necessary to measure a depth of the bone hole drilled through the bone fragments.

Many depth gauge devices exist for measuring the depth of a bone hole, however most current devices tend to be very bulky, and require the use of two hands to operate which can be very difficult depending on the type of procedure being formed. Current devices are also not particularly well suited for use in minimally invasive procedures. Minimally invasive surgical procedures, which have become more common in recent years, involve the use of a small incision that provides access to the surgical site. A cannula or access port is often used to form a pathway extending to the surgical site. Current depth gauge devices do not have a size that allows them to be used during such procedures.

Accordingly, there remains a need for improved methods and devices for measuring the dimensions of a bone hole or other lumen.

### SUMMARY OF THE INVENTION

The present invention provides various methods and devices for measuring the dimensions during surgical procedures. In one exemplary embodiment, a device for measuring the depth (or diameter) of a bone hole is provided having an indicator that is adapted to measure a dimension of a bone hole, and a positioning member disposed through the indicator for positioning the device relative to the bone hole.

The positioning member can have a variety of configurations, but in one exemplary embodiment the positioning member can have a distal end that, upon retraction of a portion of the positioning member, is adapted to radially expand to engage bone to position the device relative to a bone hole. In particular, the distal end of the positioning member can include opposed deflectable members that are adapted to expand to engage bone. In another exemplary embodiment, the opposed deflectable members can be formed on the distal end of a probe, and the positioning member can also include a spreader that is slidably disposed through the probe and that has a distal end that is adapted to expand the deflectable members upon retraction of the spreader. By way of non-limiting example, the distal end of the spreader can include an enlarged tip formed thereon for expanding the deflectable members. The enlarged tip can optionally be pointed to facilitate positioning of the device against bone.

The indicator can also have a variety of configurations, but in one exemplary embodiment, the indicator can be a depth indicator. The indicator can also be a diameter indicator. The indicator is in the form of a barrel and a sleeve that is slidably disposed over the barrel. The barrel can include indicia formed thereon to indicate a depth of the sleeve when the sleeve is disposed within a bone hole. In an exemplary embodiment, the barrel can be mated to the probe of the positioning member. While a variety of techniques can be used to mate the barrel and the probe, in one embodiment the probe can be threadably mated to the barrel. The device can also include one or more ring-shaped handles adapted for one-handed use located on one or more of the various components. In one exemplary embodiment, the barrel, the sleeve, and the spreader can each include a handle formed on a proximal end thereof.

Methods for measuring the depth of a bone hole are also disclosed. One exemplary method includes inserting a depth gauge device through a bone hole, retracting a portion of the device to radially expand a distal end of the device, retracting the device to cause the radially expanded distal end to engage bone, and distally advancing a depth indicator into the bone hole to measure the depth of the bone hole. The device can also be adapted for use with one hand. Other methods include using the device to measure the diameter of a cavity.

### BRIEF DESCRIPTION OF THE DRAWINGS

The invention will be more fully understood from the following detailed description taken in conjunction with the accompanying drawings, in which:

FIG. 1 is a side, transparent view of one exemplary embodiment of a depth gauge device;

FIG. 2A is a side, transparent view of one exemplary embodiment of a probe of the depth gauge device shown in FIG. 1;

FIG. 2B is a side view of a distal end of the probe shown in FIG. 2A;

FIG. 3 is a side view of one exemplary embodiment of a spreader of the depth gauge device shown in FIG. 1;

FIG. 4A is a side, transparent view of one exemplary embodiment of a barrel of the depth gauge device shown in FIG. 1;

FIG. 4B is a top view of a proximal end of the barrel shown in FIG. 4A;

FIG. 5 is a side, transparent view of one exemplary embodiment of a measuring sleeve of the depth gauge of FIG. 1;

FIG. 6A is a schematic showing a bone hole;

FIG. 6B is a schematic showing the depth gauge device of FIG. 1 inserted through the bone hole shown in FIG. 6A;

FIG. 6C is a schematic showing a distal end of the depth gauge device of FIG. 1 expanded, and showing the depth indicator retracted to engage bone;

FIG. 6D is a schematic showing a depth indicator of the depth gauge device shown in FIG. 1 being used to measure a depth of the bone hole from the tissue surface;

FIG. 7A is a schematic showing one exemplary embodiment of a gauge device inserted through a cavity; and

FIG. 7B is a schematic showing the device of FIG. 7A expanded and used to measure a diameter of a cavity.

### DETAILED DESCRIPTION OF THE INVENTION

Various exemplary methods and devices are provided for measuring the depth of a bone hole or other pathway. A person skilled in the art will appreciate that, while the methods and devices are described for use in measuring a bone hole, the methods and devices can be used in a variety of other surgical procedures for obtaining a variety of measurements, e.g., the diameter of a cavity, such as the intermedullary portion of a bone.

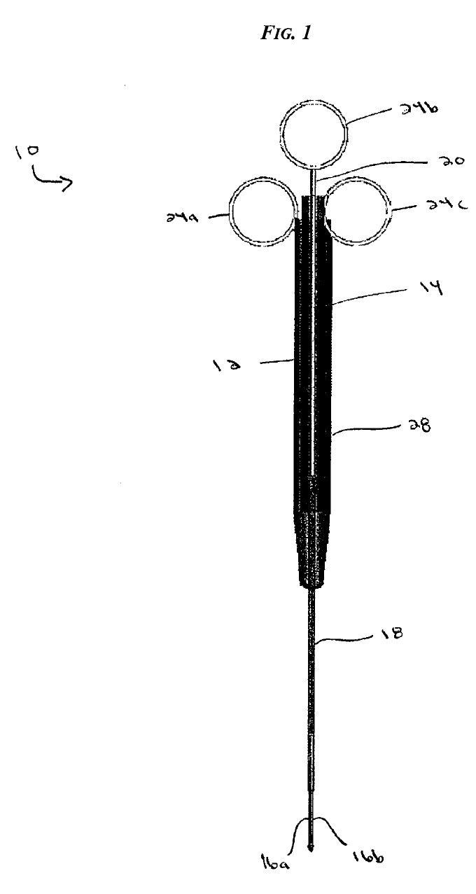

FIG. 1 illustrates one exemplary embodiment of a depth gauge device 10, and as shown the device 10 generally includes a depth indicator 12 that is adapted to measure a depth of a bone hole and a positioning member 14 disposed through the depth indicator 12. While the depth indicator 12 and the positioning member 14 can each have a variety of configurations, in the illustrated embodiment the positioning member 14 includes a probe 18 having a spreader 20 slidably disposed therein, and the depth indicator 12 includes a barrel 26 slidingly disposed within a measuring sleeve 28. In use, the device 10 can be inserted through a bone hole and the spreader 20 can be retracted to radially expand a distal end of the probe 18. The sleeve 28 can then be advanced into the bone hole to measure the depth of the bone hole.

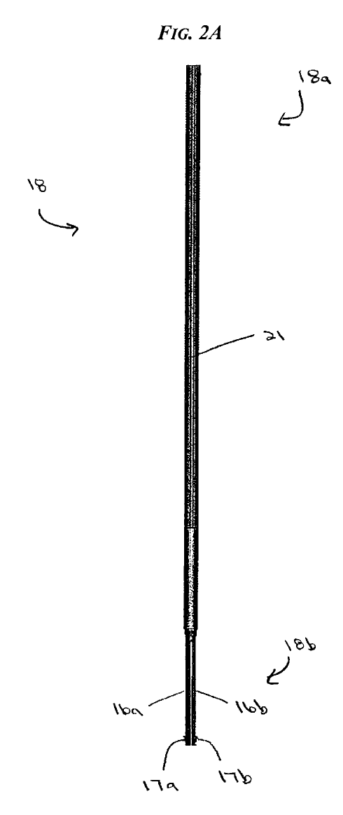

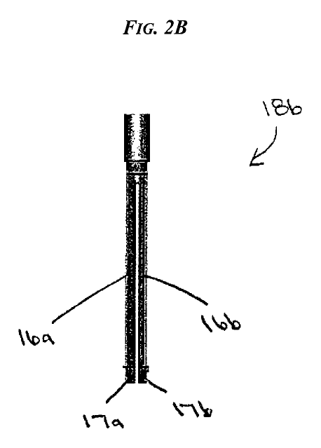

The probe 18 of the positioning member 14, which is shown in more detail in FIGS. 2A-2B, can have a variety of configurations, but preferably it is adapted to engage a bone hole to facilitate positioning of the device 10. In an exemplary embodiment, as shown, the probe 18 is in the form of an elongate shaft having an inner lumen 21 extending therethrough between the proximal and distal ends 18a, 18bthereof. The proximal end 18aof the probe 18 can be adapted to mate with the barrel 28 of the depth indicator 12 to connect the positioning member 14 to the depth indicator 12. Various mating techniques can be used to mate the probe 18 and the barrel 28, and exemplary mating techniques include threads, a dovetail connection, an interference or a snap fit, a magnetic attachment, adhesives, etc. In an exemplary embodiment, the proximal end 18aof the probe 18 includes threads formed therearound to mate with threads formed within the barrel 28. While the probe 18 and the barrel 26 are shown as separate components, they can optionally be formed as a single integrated piece. The proximal end 18aof the probe 18 can also include a handle for gripping the device. While the handle can have any configuration, in the illustrated embodiment the handle is a ring-shaped member 24b that is adapted to receive a finger therethrough. This allows a surgeon to operate the device with one hand, which is particularly advantageous when performing minimally invasive procedures.

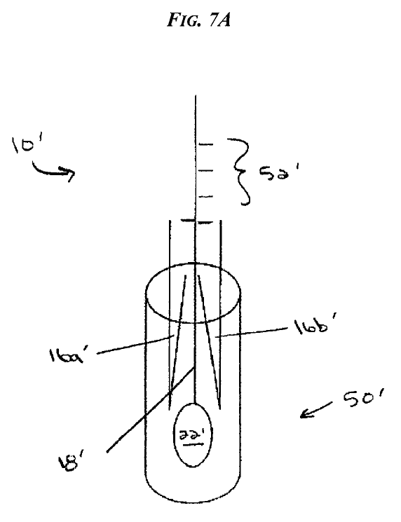

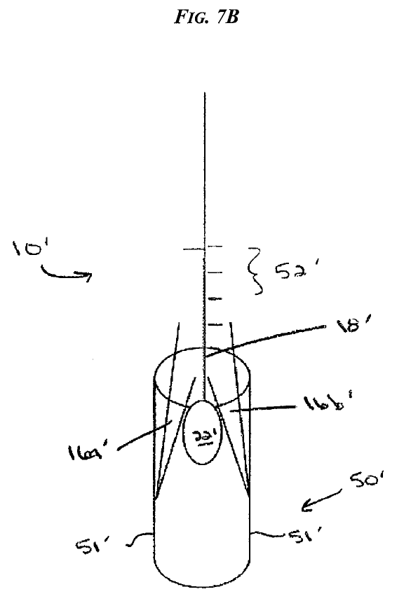

The distal end 18bof the probe 18 can also have a variety of configurations, but in one exemplary embodiment the distal end 18bincludes deflectable members 16a , 16b formed thereon. The deflectable members 16a , 16b can be adapted to radially expand to engage bone, as will be discussed in more detail below. While the deflectable members 16a , 16b can have a variety of shapes and sizes, in one exemplary embodiment, as shown in FIG. 2B, the deflectable members 16a , 16b are in the form of opposed legs that are separated by a slit extending therebetween. In another exemplary embodiment, as shown in FIG. 7A, the deflectable members 16a ′, 16b ′ are in the form of calipers or hooks separated at a certain angle by a slit extending therebetween. While the deflectable members 16a , 16b (as well as deflectable members 16a ′, 16b ′) are shown to be opposed from one another, the distal end 18b of the probe 18 can optionally have a single deflectable member. Referring back to FIG. 2B, the distal-most end of each deflectable member 16a , 16b can include an enlarged flange 17a, 17b formed thereon to facilitate engagement of the bone hole. In an exemplary embodiment, each flange 17a, 17b tapers in the distal direction to facilitate insertion thereof through a bone hole.

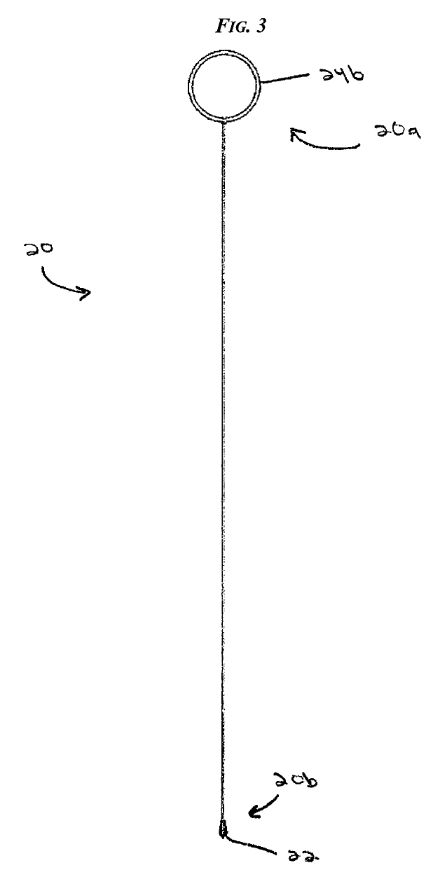

As noted above, the positioning member 14 can also include a spreader 20 that is slidably disposed through the probe 18. The spreader 20 can have a variety of configurations, however, in the exemplary embodiment shown in FIG. 3, the spreader 20 is in the form of an elongate shaft having proximal and distal ends 20a, 20b. The spreader 20 can be a single elongate piece, but in an exemplary embodiment it is formed from two separate pieces that are removably mateable to one another. One skilled in the art will appreciate that the two pieces of the spreader 20 can be mated to one another by a variety of mating techniques, such as threads, a dovetail connection, an interference or snap fit, a magnetic attachment, adhesives, etc.

The distal end 20b of the spreader 20 is preferably adapted to expand the deflectable members 16a , 16b on the probe 18, and thus in an exemplary embodiment the distal end 20b can include an enlarged tip 22 formed thereon. The enlarged tip 22 can have a variety of configurations, however as shown in FIG. 3, the enlarged tip 22 is substantially diamond-shaped such that it has a proximal portion that tapers in a proximal direction, and a distal portion that tapers in a distal direction. The enlarged tip 22 can also include a pointed tip to facilitate positioning of the device 10 against bone, and in particular to provide tactile feedback to the surgeon. As shown in FIG. 7A, the enlarged tip 22′ can have a substantially spherical shape. A person having skill in the art will appreciate that the tip can have a variety of other shapes and sizes.

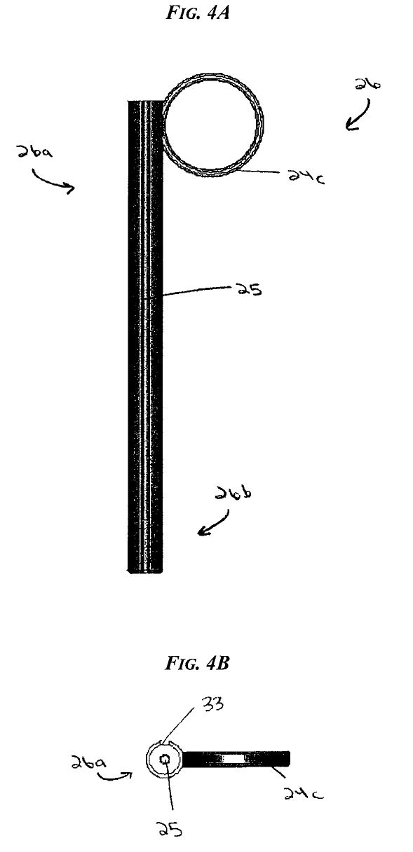

As noted above, with respect to FIG. 1, the device 10 can also include a depth indicator 12 having a barrel 26 and a sleeve 28. While the barrel 26 can have a variety of configurations, FIG. 4A illustrates an exemplary embodiment of the barrel 26. As shown, the barrel 26 is in the form of an elongate shaft having an inner lumen 25 extending therethrough between the proximal and distal ends 26a, 26b thereof. The proximal end 26a can include a handle, such as a ring-shaped handle 24a similar to that previously discussed above with respect to FIG. 3. As shown in FIG. 4B, the proximal end 26a of the barrel 26 can also include a notch or slot 33 formed therein to accept a portion of the ring-shaped handle 24a formed on the measuring sleeve 28, as will be discussed in more detail below. The notch 33 preferably extends along a portion of the barrel 26 to allow the measuring sleeve 28 to slidably move relative to the barrel 26.

The barrel 26 can also include various features formed thereon to facilitate alignment of the barrel 26 with the measuring sleeve 28, as well as to assist the surgeon in measuring the depth of the bone hole. In one embodiment, the barrel 26 can have at least one groove (not shown) formed on all or a portion of the outer surface thereof and extending between the proximal and distal ends 26a, 26b of the barrel 26. The groove can be adapted to receive a pin member 34 formed within the inner lumen 31 of the sleeve 28 to prevent rotation of the sleeve 28 relative to the barrel 26. A person having skill in the art will appreciate that a variety of other techniques can be used to prevent rotation between the sleeve 26 and the barrel 28, or to otherwise align the two components. The barrel 26 can also include features to facilitate measuring the depth of a bone hole. In one exemplary embodiment, the outer surface of the barrel 26 includes indicia formed thereon, and is adapted to indicate a depth of the sleeve 26, which corresponds to the depth of the bone hole. In another exemplary embodiment, as will be discussed in more detail below with respect to FIGS. 7A-7B, the indicia 52′ formed on the outer surface of the barrel can be calibrated to correspond to the diameter of a cavity 50′, such as the intermedullary portion of bone.

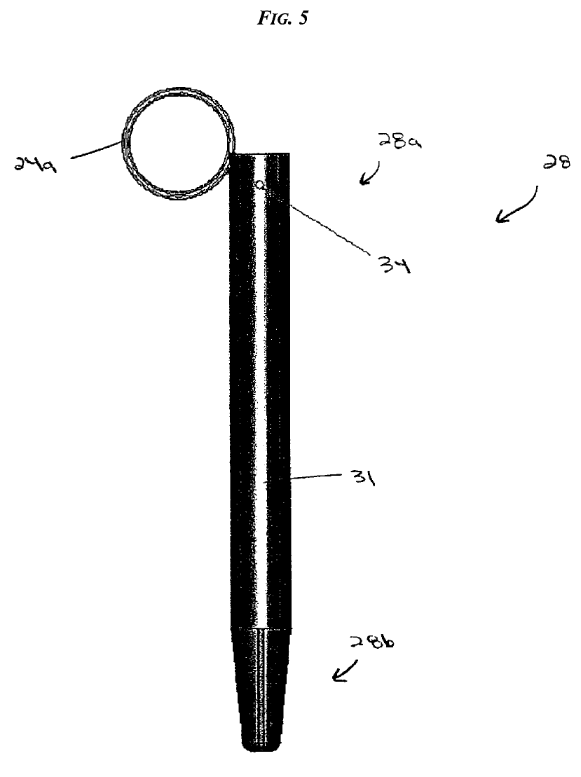

The exemplary measuring sleeve 28 is shown in more detail in FIG. 5. While the measuring sleeve 28 can have a variety of configurations, as shown the exemplary measuring sleeve 28 is in the form of an elongate shaft having an inner lumen 31 extending between the proximal and distal ends 28a, 28b thereof. The proximal end 28a can include a ring-shaped handle 24a , similar to that as discussed above with respect to FIGS. 3-4B. The distal end 28b of the measuring sleeve 28 can be configured to facilitate insertion into the bone hole, and in one embodiment it can include a slight distal taper.

As previously discussed, the sleeve 28 can include at least one mating feature, such as a pin 34, that is adapted to extend into the groove formed on the outer surface of the barrel 26. As shown in FIG. 5, the pin 34 is formed adjacent to the proximal end 28a of the sleeve 28, and it extends into the inner lumen 31. In use, the pin 34 allows the measuring sleeve 28 to be freely slidable, yet at the same time nonrotatable, with respect to the barrel 26.

Referring back to FIG. 1, when the device 10 is assembled the spreader 20 is disposed through the probe 18 such that the enlarged distal tip 22 on the spreader 20 is positioned distal of the deflectable members 16a , 16b on the probe 18. The probe 18 and spreader 20, which can form the positioning member 14, are disposed through the depth indicator 12. In particular, the spreader 20 can be threadably mated to the barrel 26, and the sleeve 28 can be slidably disposed around the barrel 26.

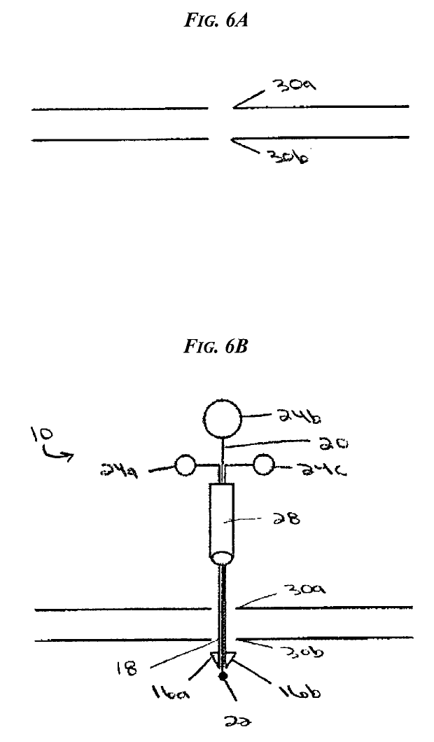

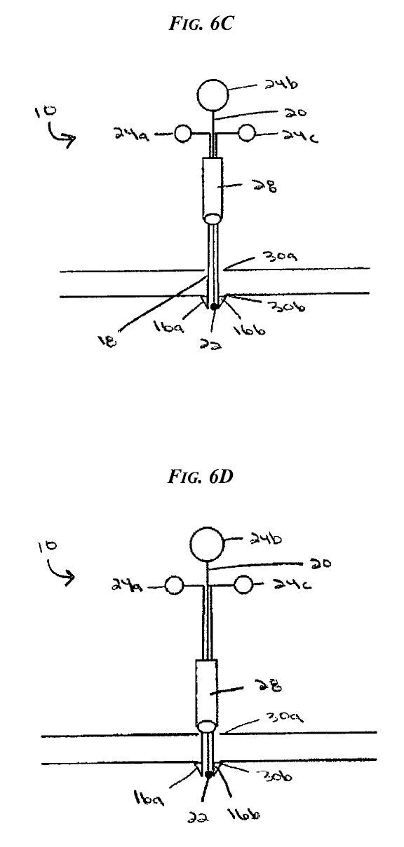

FIGS. 6A-6D illustrate an exemplary method for measuring the depth of a bone hole using the depth gauge 10 as disclosed herein. A person having skill in the art will appreciate that a variety of other devices can be used to perform the exemplary method, and that device 10 is merely shown for illustration purposes. As shown in FIG. 6A, the surgeon first forms upper and lower holes or bores 30a , 30b in the patient's bone. Upon formation of the bone holes 30a , 30b , the surgeon grasps the depth gauge 10 by placing his fingers through the ring-shaped handles 24a, 24b, 24c. With the center ring-shaped handle 24b at a distal most position such that the spreader 20 is fully inserter through the probe 18, the surgeon then inserts the depth gauge 10 into the upper and lower bone holes 30a, 30b, as shown in FIG. 6B. The pointed distal end of the spreader 20 can be used to facilitate positioning of the device 10 through the bone holes. The surgeon can then pull the spreader 20 in a proximal direction to retract it by moving the finger placed in the ring-shaped handle 24b. As a result, the enlarged tip 22 of the spreader 20 will radially expand the deflectable members 16a, 16b on the distal end of the probe 18, as shown in FIG. 6C. By way of non-limiting example, the deflectable members 16a, 16b can radially expand from about 0.5 mm to about 20.0 mm.

Following the expansion of the deflectable members 16a, 16b by the spreader 20, the device 10 can be retracted to cause the deflectable members 16a, 16b to engage the bone adjacent to the distal-most hole 30b, thereby maintaining the device 10 in a substantially fixed position relative to the bone. The surgeon can then advance the measuring sleeve 28 towards the upper bone hole 30a until the sleeve 28 abuts against the upper bone hole 30a. The finger loops 24a, 24b, 24c can be used to move the sleeve 28 relative to the barrel 26. The indicia on the barrel 26 can then be viewed to determine that depth of the sleeve 28, thereby indicating the depth of the bone holes 30a, 30b .

To effect removal of the depth gauge 10 from the patient, the surgeon can push the center ring 24b in a distal direction such that the distal end of the probe 18 is moved out from between the deflectable members 16a, 16b, thereby allowing the deflectable members 16a, 16b to return to an un-deflected state.

FIGS. 7A-7B illustrate a method for measuring the diameter of a cavity, such as the intermedullary portion of a bone, and an exemplary embodiment of a device useful for doing so. Similar to the technique discussed above with respect to FIGS. 6A-6D, the surgeon inserts the depth gauge 10′ into the cavity 50′ as shown in FIG. 7A. The pointed distal end of the spreader (not shown) can help to facilitate insertion of the device 10′ into the cavity 50′. Once inserted within the cavity 50′ , as shown in FIG. 7B, the surgeon can pull the spreader in a proximal direction, such that the enlarged tip 22′ of the spreader will radially expand the deflectable members 16a ′, 16b ′ on the distal end of the probe 18′ until the deflectable members 16a ′, 16b ′ contact the inner surface of the cavity 51′. Similar to the embodiment discussed above, and by way of non-limiting example, the deflectable members 16a′, 16b′ can radially expand from about 0.5 mm to about 20.0 mm.

Once the deflectable members 16a′, 16b′ have contacted the inner surface of the cavity 51′, the surgeon can view the indicia 52′ on the barrel to determine the amount of retraction of the probe 18′. The amount of probe retraction can be calibrated to correspond to cavity diameter measurements.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A surgical measuring device, comprising:
an indicator adapted to measure a dimension of a bone hole; and
a positioning member disposed through the indicator and having a distal end that, upon retraction of a portion of the positioning member, is adapted to radially expand to engage bone to position the positioning member relative to a bone hole, thereby allowing the dimension of the bone hole to be measured using the indicator.

2. The device of claim 1, wherein the distal end of the positioning member includes opposed deflectable members that are adapted to expand to engage bone.

3. The device of claim 2, wherein the positioning member comprises a probe having the deflectable members formed on the distal end thereof, and a spreader slidably disposed through the probe and having a distal end that is adapted to expand the deflectable members upon retraction of the spreader.

4. The device of claim 3, wherein the distal end of the probe includes an enlarged tip formed thereon for expanding the deflectable members.

5. The device of claim 4, wherein the enlarged tip is pointed to facilitate positioning of the positioning member relative to bone.

6. The device of claim 4, wherein the spreader is movable between a first distal position in which the enlarged tip is positioned distal to the deflectable members, and a second proximal position in which the enlarged tip is positioned between the deflectable members to expand the deflectable members.

7. The device of claim 3, wherein the spreader includes proximal and distal portions that are removably mated to one another.

8. The device of claim 3, wherein the spreader includes a handle formed on a proximal end thereof.

9. The device of claim 8, wherein the handle comprises a ring-shaped member.

10. The device of claim 3, wherein the probe is adapted to mate to a portion of the depth indicator.

11. The device of claim 1, wherein the indicator comprises a barrel and a sleeve slidably disposed over the barrel.

12. The device of claim 11, wherein the barrel includes a plurality of indicia formed thereon and adapted to indicate a depth of the sleeve when the sleeve is disposed within a bone hole, the depth of the sleeve being indicative of the depth of the bone hole.

13. The device of claim 11, wherein the positioning member comprises a probe mated to the barrel and having the deflectable members formed on the distal end thereof, and a spreader slidably disposed through the probe and having a distal end that is adapted to expand the deflectable members.

14. The device of claim 13, wherein the probe is threadably mated to the barrel.

15. The device of claim 13, wherein the barrel, the sleeve, and the spreader each include a handle formed on a proximal end thereof.

16. The device of claim 15, wherein each handle comprises a ring-shaped member.

17. The device of claim 1, wherein the indicator is a depth indicator.

18. The device of claim 1, wherein the indicator is a diameter indicator.

19. A method for measuring the depth of a bone hole, comprising:
inserting a depth gauge device through a bone hole;
retracting at least a portion of the device to radially expand a distal end of the device and to cause the radially expanded distal end to engage bone; and
distally advancing a depth indicator into the bone hole to measure the depth of the bone hole.

20. The method of claim 19, wherein the device is inserted through a minimally invasive incision.

21. The method of claim 19, wherein distally advancing the depth indicator into the bone hole comprises sliding a sleeve over a barrel having indicia thereon, and measuring a depth of the bone hole based on the depth of the sleeve as indicated by the indicia.

22. The method of claim 19, wherein the step of retracting at least a portion of the device comprises retracting a spreader slidably disposed through a probe to radially expand opposed deflectable members formed on the distal end of the probe.

23. The method of claim 19, wherein the device is adapted to be used with one hand.

24. A method for measuring the diameter of a cavity, comprising:
inserting a gauge device into a cavity;
retracting at least a portion of the device to radially expand a distal end of the device and to cause the radially expanded distal end to engage an inner surface of the cavity; and
calibrating the device such that the amount of retraction corresponds to the diameter of the cavity.
